(19) United States
(12) Patent Application Publication
(10) Pub. No.: US 2026/0174456 A1
(43) Pub. Date: Jun. 25, 2026

(54) ADAPTER DEVICE, DISASSEMBLABLE MEDICAL SHAFT INSTRUMENT AND METHOD FOR CLEANING THE DISASSEMBLABLE MEDICAL SHAFT INSTRUMENT AND ASSEMBLY METHOD

(71) Applicant: KARL STORZ SE & Co. KG, Tuttlingen (DE)

(72) Inventors: Monja ILKA, Tuttlingen (DE); Daniel KÄRCHER, Tuttlingen (DE); Robin MERZ, Tuttlingen (DE); Claus KRAMER, Tuttlingen (DE); Michael EGLE, Tuttlingen (DE)

(21) Appl. No.: 19/425,495

(22) Filed: Dec. 18, 2025

(30) Foreign Application Priority Data
Dec. 20, 2024 (DE) 10 2024 139 321.1

(51) Int. Cl. A61B 17/29; A61B 17/00

(52) U.S. Cl. CPC A61B 17/2909; A61B 2017/0046; A61B 2017/00477; A61B 2017/292

(57) ABSTRACT

The present invention provides an adapter device and a disassemblable medical shaft instrument equipped therewith, a method for cleaning the disassemblable medical shaft instrument and a method for assembling the adapter device to provide the disassemblable medical shaft instrument. The adapter device (10) is designed for arrangement in a disassemblable medical shaft instrument (1) comprising a shaft (2), a transmission rod (40) that can move in the shaft (2) and a handling apparatus (3) comprising a housing part (30) and an actuating part (31) which is movable relative to the housing part (30). The adapter device (10) has a transmission adapter (5) designed for releasably coupling to the actuating part (31) and to the transmission rod (40), and an adapter sleeve (6) designed for releasably coupling to the shaft (2) and the housing part (30). The adapter sleeve (6) has an axial bore (64, 65, 66, 67) in which a distal coupling portion (52) of the transmission adapter (5) is arranged, and wherein the transmission adapter (5) is movable in a translational manner relative to the adapter sleeve (6). According to the invention, a proximal portion (67) of the axial bore (64, 65, 66, 67) of the adapter sleeve (6) is frustoconical and widens toward the proximal end of the axial bore (64, 65, 66, 67), wherein the frustoconical proximal portion (67) is designed to releasably receive a conical nozzle (21) of a flushing device (20).

The invention relates to an adapter device and to a disassemblable medical shaft instrument equipped therewith. Furthermore, the invention relates to a method for cleaning the disassemblable medical shaft instrument and to a method for assembling the adapter device for providing the disassemblable medical shaft instrument.

It is known from the prior art that medical, in particular surgical, instruments are designed to be disassembled in order to clean and sterilize them so that they can be reused. A typical shaft instrument has a shaft, a handling apparatus at the proximal end of the shaft, and a tool insert with a tool at the distal end of the shaft and a transmission rod extending through the shaft to couple the tool to the handling apparatus. With a disassemblable shaft instrument, the shaft can usually be separated from the handling apparatus and, if necessary, also from the tool insert. The handling apparatus has a housing part that is connected to the shaft and an actuating part that is movable relative thereto and is coupled to the transmission rod such that a relative movement between the actuating part and the housing part of the handling apparatus causes a translational movement of the transmission rod within the shaft in order to actuate the tool at the distal end of the shaft.

The transmission rod has a coupling element at its proximal end for releasably coupling to the actuating part of the handling apparatus so that the transmission rod can be separated from the handling apparatus and, if the tool insert can also be separated from the shaft, can be pulled toward the distal end out of the shaft. Since the shaft diameter and cross section of the transmission rod correspond, the size of the coupling element in disassemblable shaft instruments must not exceed the cross section of the transmission rod. However, shaft instruments for minimally and microinvasive procedures have shafts that are as thin as possible so that the cross section of the transmission rods and thus also the size of the coupling elements must be accordingly small. However, the size of the coupling element determines the actuating force that can be transmitted from the actuating part to the transmission rod. Therefore, for different shaft diameters, different handling apparatuses or handling apparatuses comprising devices that allow for appropriate adjustments to different shaft diameters and coupling elements of different sizes must be used.

DE 10 2012 200 073 A 1 describes a medical instrument that allows a handling apparatus to be combined with different shafts and different transmission rods. For this purpose, a transmission adapter is provided, which is coupled to the coupling element at the proximal end of the transmission rod on the one hand and to the actuating part of the handling apparatus on the other. For coupling to the transmission rod, the transmission adapter has gripping jaws that can be pivoted in an articulated fashion and are pre-tensioned by an elastic element into a position in which the gripping jaws hold the coupling element of the transmission rod by means of a form fit and a force fit. The transmission adapter is movably arranged in a sleeve-like shaft adapter, which is releasably coupled on the one hand to the housing part of the handling apparatus and to the shaft on the other.

The transmission adapter and the shaft adapter allow a thin shaft with a diameter of less than 5 mm, e.g., 3.5 mm, and a tool insert with a correspondingly thin transmission rod to be coupled to a conventional handling apparatus. However, this method has the disadvantage of requiring many components and a corresponding amount of assembly effort. This is because, during reprocessing of the disassembled instrument, a rinsing port must be integrated for rinsing the thin shaft, which is usually arranged at the proximal end of the shaft at the interface to the handling apparatus. Axial rinsing from the proximal side of the transmission adapter is not possible because, due to the open design of the transfer adapter required for the articulated gripping jaws, the rinsing pressure arriving at the distal end of the shaft is insufficient for reliable and safe reprocessing.

DE 10 2016 118 304 A1 discloses a safety apparatus for preventing overloading of the mechanics of microinvasive instruments. For this purpose, it comprises a transmission apparatus that is movable within the shaft and comprises stop surfaces for limiting the movement of this transmission device.

DE 10 2022 107 371 A1 discloses a shaft assembly comprising a tubular shaft and movable operating elements as well as a handle assembly comprising an actuating element, which can be connected to one another in a reversibly releasable manner and so as to be rotatable about the longitudinal axis. Flushing liquid can be introduced into the annular gap between the shaft and the rod element via an axial flushing port. The handle assembly has a head with distal and proximal parts, wherein the shaft is fastened to the distal head part for conjoint rotation therewith and central openings and bores axially pass through both head parts.

DE 20 2021 102 135 U1 discloses a surgical instrument comprising a tubular shaft with at least one movable operating element, which is moved via a displaceable rod element by actuating a handle actuating element. Said instrument has an axial flushing port for introducing flushing liquid into the annular space between the shaft and rod element.

US 2017/0367722 A1 (DE 10 2012 022 573A1 ) teaches a medical instrument whose shaft and handling apparatus can be disassembled in order to clean and sterilize them, and in which different transmission apparatuses can be combined with a standard handling apparatus. The core of the solution is a transmission adapter that is arranged between the handling apparatus and the proximal end of the transmission apparatus and allows for a releasable mechanical connection by means of two couplings. This makes it easier to adapt to different shaft diameters and coupling sizes.

Proceeding from this prior art, the object of the present invention is to provide an improved adapter concept.

This object is achieved by an adapter device having the features of claim 1.

Embodiments of the present invention are based on the idea of providing a transmission adapter which can be coupled at one end to the proximal end of a transmission rod in a shaft for a medical shaft instrument and at the other end to a handling apparatus for a medical shaft instrument. In particular, a transmission adapter is provided each time for a plurality of different transmission rods or for a plurality of transmission rods with different couplings at their proximal ends. A distal end or coupling at the distal end of each transmission rod is adapted to a specific design of the proximal end of a transmission rod; the proximal ends of the transmission adapters are identical and, in particular, adapted to a standard handling apparatus.

A transmission adapter for a medical instrument comprising a shaft, a transmission rod that can move within the shaft, and a handling apparatus comprising a housing part that can be mechanically rigidly coupled to a proximal end of the shaft and an actuating part that is movable relative to the housing part, comprises a first coupling for releasably mechanically coupling to the actuating part of the handling apparatus and a second coupling for releasably or non-releasably mechanically coupling to a proximal end of the transmission rod.

The transmission adapter is in particular provided and configured as a component of a medical shaft instrument or together with other components to form a medical shaft instrument. The shaft is straight or curved, partially or completely rigid, or partially or completely elastic or plastically deformable. The transmission rod can move in a translational and/or rotational manner within the shaft to transmit a tensile and/or compressive force and/or torque between the handling apparatus and a distal end of the shaft, in particular a tool at the distal end of the shaft.

The additional object of providing an improved disassemblable medical shaft instrument is achieved by the disassemblable medical shaft instrument having the features of independent claim 10.

Another object consists in improving cleaning of a disassemblable medical shaft instrument, and is achieved by the method having the features of independent claim 11.

Yet another object of assembling the adapter device in a disassemblable medical shaft instrument is achieved by the method having the features of independent claim 12.

Developments or preferred embodiments of the adapter device, the shaft instrument, the cleaning method and the assembly method are described in the respective dependent claims.

An adapter device according to the invention is designed for arrangement in a disassemblable medical shaft instrument, which comprises a shaft, a transmission rod that is movable within the shaft and a handling apparatus comprising a housing part and an actuating part that is movable relative to the housing part. According to a first embodiment, the adapter device has a transmission adapter designed for releasably coupling to the actuating part and to the transmission rod, and an adapter sleeve designed for releasably or non-releasably coupling to the shaft and to the housing part.

The adapter sleeve has an axial bore in which a distal coupling portion of the transmission adapter is arranged. The transmission adapter can move in a translational manner relative to the adapter sleeve. According to the invention, a proximal portion of the axial bore of the adapter sleeve is frustoconical and widens toward the proximal end of the axial bore, wherein the frustoconical proximal portion, which is hereinafter also referred to as the conical portion for short, is designed for releasably receiving a conical nozzle of a flushing device.

By means of corresponding frustoconical shapes of the conical portion and the nozzle, the nozzle can be clampingly received in the conical portion for cleaning purposes, and thus sealed. This enables safe reprocessing, in particular also of thin shafts, by axially supplying a flushing medium from the proximal side of the adapter device so that sufficient flushing medium can exit at the distal end of the shaft. The adapter device is compatible with existing mass-produced instrument components such as handling apparatuses, transmission rods and shafts, in particular also those with a diameter of less than 5 mm, e. g., 3.5 mm, and has a simple design in terms of manufacturing and assembly.

The adapter device according to the invention comprises two adapters as mechanical connecting pieces between the different interfaces of the instrument components when the existing coupling elements have different dimensions. That is, a distal coupling element of the transmission adapter is designed for releasably coupling to the proximal coupling element of the transmission rod, and a proximal coupling element of the transmission adapter is designed for releasably coupling to a coupling element of the actuating part of the handling apparatus. The adapter sleeve provides coupling elements for releasably or non-releasably coupling at one end to the proximal end of the shaft and at the other end to the housing part of the handling apparatus.

According to a further embodiment of the adapter device according to the invention, a proximal end portion of the adapter sleeve has an external thread which consequently surrounds the conical portion. This external thread is designed for releasably connecting to an internally threaded tube portion of the flushing device surrounding the nozzle so that when the internally threaded tube portion is screwed onto the external thread of the adapter sleeve, the nozzle of the flushing device is clampingly received in the conical portion. The screw connection ensures secure sealing of the clamp connection even at higher flushing pressures.

Advantageously, according to a further embodiment of the adapter device according to the invention, the proximal frustoconical proximal portion can be shaped in accordance with a standardized connecting system. The Luer system (internationally standardized in ISO standard ISO 80369) may be particularly preferred, as its use is common in the medical field, e. g., for cannulas and syringes, etc. The frustum shape of the male and female connecting pieces has a cone angle of 3.44°. The plug connection without a screw thread is also called a “Luer Slip” and the plug connection with a screw thread is called a “Luer Lock.”

Another embodiment of the adapter device according to the invention relates to the distal coupling portion of the transmission adapter having an axial receiving bore and at least one engagement opening in the radial direction that is associated therewith. The adapter device has at least one engagement slide with a plug portion which is arranged in the engagement opening and extends into the receiving bore in the transmission adapter. The plug portion has a recess designed to receive a proximal coupling element of the transmission rod. The adapter sleeve has at least one longitudinal opening in a central portion, which is associated with the axial bore and is longer than the plug portion, which extends through the longitudinal opening for arrangement in the engagement opening in the distal coupling portion. Thus, the engagement slide together with the transmission adapter can be moved in a translational manner relative to the adapter sleeve.

The plug portion can preferably be designed for positive insertion into the engagement opening, and the recess can also be designed for positive reception of the proximal coupling element. Furthermore, the width of the plug portion can correspond to the width of the longitudinal opening so that the engagement slide is securely guided in the longitudinal opening during the translational movement with the transmission adapter.

According to an advantageous embodiment, the engagement slide can have a slider portion from which the plug portion extends. The adapter sleeve can have a sliding surface in the central portion around the longitudinal opening, on which surface the slider portion of the engagement slide rests such that it can move in a translational manner. The length and width of the slider portion are of such a size that the longitudinal opening is always covered by the slider portion. In this way, the engagement slide seals the axial bore of the adapter sleeve.

Preferably, according to a further embodiment, the adapter device can have two engagement slides, two engagement openings in the distal coupling portion of the transmission adapter and two longitudinal openings in the central portion of the adapter sleeve, each arranged opposite one another in the radial direction.

To secure the engagement slide(s), according to a further embodiment, said slide(s) has/have at least one circumferentially extending receiving groove on an outer side facing away from the plug portion, in which groove an elastic O-ring is received that surrounds the central portion of the adapter sleeve.

Furthermore, according to yet another embodiment, the distal coupling portion may have an edge shape with at least one axially parallel guide surface for axially guiding the transmission adapter in the adapter sleeve.

According to a further embodiment, a distal portion of the axial bore is designed to releasably receive a proximal end of the shaft in order to couple the adapter sleeve of an adapter device according to the invention to a shaft. The distal portion is associated with a connecting portion of the axial bore of reduced diameter, which is designed for the passage of the transmission rod. In this case, a sealing groove can be formed in the connecting portion adjacently to the distal portion, in which groove a sealing ring is arranged to seal the transmission rod.

According to a further embodiment, additional coupling of the adapter sleeve to the housing part of the handling apparatus can involve a distal end portion of the adapter sleeve being cylindrical and delimited by a collar, which serves as a stop for arrangement in the housing part.

In a first embodiment, a disassemblable medical shaft instrument according to the invention comprises a handling apparatus comprising a housing part and an actuating part that is movable relative thereto, a shaft, a transmission rod that is movable within the shaft and an adapter device according to the invention. The adapter device comprises a transmission adapter for releasably coupling to the actuating part and to the transmission rod, and an adapter sleeve for releasably or non-releasably coupling to the housing part and the shaft. In this arrangement, a distal coupling portion of the transmission adapter is arranged in an axial bore of the adapter sleeve, and the transmission adapter is movable in a translational manner relative to the adapter sleeve. The use of the adapter device according to the invention in the shaft instrument enables safe reprocessing, in particular also of thin shafts, by axially supplying a flushing medium from the proximal side of the adapter device so that sufficient flushing medium can exit at the distal end of the shaft. The design is simplified, making the shaft instrument easy to manufacture and assemble.

A method according to the invention relates to the process of cleaning a disassemblable medical shaft instrument according to the invention, which comprises an adapter device according to the invention. The cleaning process involves the steps of:
Decoupling the transmission adapter from the actuating part and decoupling the adapter sleeve from the housing part, and
Separating the transmission adapter that is coupled to the transmission rod and, in particular, the adapter sleeve that is coupled to the shaft from the handling apparatus,
Decoupling the transmission adapter from the transmission rod and removing the transmission rod from the shaft,
Arranging the conical nozzle of a flushing device in the frustoconical proximal portion of the axial bore of the adapter sleeve, and
Supplying a rinsing medium by means of the rinsing device through the nozzle into the axial bore of the adapter sleeve and the shaft coupled thereto so that the flushing medium exits at the distal end of the shaft, thereby cleaning the medical shaft instrument.

Another method according to the invention relates to the assembling of an adapter device according to the invention for providing a disassemblable medical shaft instrument according to the invention, and comprises the following steps:
Providing the transmission adapter and adapter sleeve and providing the handling apparatus comprising the housing part and the actuating part that is movable relative thereto and the shaft with the transmission rod that can move within the shaft,
Arranging the distal coupling portion of the transmission adapter in the axial bore of the adapter sleeve,
Releasably coupling the transmission adapter to the transmission rod and releasably or non-releasably coupling the adapter sleeve to the shaft,
Arranging the transmission adapter that is coupled to the transmission rod and the adapter sleeve that is coupled to the shaft in the handling apparatus,
Releasably coupling the adapter sleeve to the housing part and releasably coupling the transmission adapter to the actuating part.

According to another embodiment of the assembly method, releasably coupling the transmission adapter to the transmission rod further involves the steps of:

Aligning the at least one engagement opening associated with the axial receiving bore in the distal coupling portion with the at least one longitudinal opening in the central portion of the adapter sleeve when arranging the distal coupling portion of the transmission adapter in the axial bore of the adapter sleeve,
Arranging the proximal coupling element of the transmission rod in the receiving bore in the distal coupling portion,
providing the at least one engagement slide,
Inserting the plug portion of the engagement slide through the longitudinal opening (62) and the engagement opening into the receiving bore, and
Receiving the proximal coupling element of the transmission rod in the recess in the plug portion.

According to yet another embodiment of the assembly method, releasably coupling the transmission adapter to the transmission rod further comprises the step of arranging an elastic O-ring in the receiving groove on the outer side of the engagement slide.

Further embodiments, as well as some of the advantages associated with these and other embodiments, are made apparent and better understood from the following detailed description with reference to the accompanying figures. Objects or parts thereof which are substantially the same or similar may be provided with the same reference signs. The figures are merely a schematic representation of an embodiment of the invention. The drawings, the description, and the claims contain numerous features in combination. It is understood that the features mentioned above and those to be explained below can be used not only in the combination specified in each case, but also in other combinations or on their own, without departing from the scope of the present invention.

### In the Drawings:

FIG. 1 shows a lateral view of a disassemblable shaft instrument from the prior art comprising a radial flushing port,

FIG. 2 shows a schematic view of a handling apparatus comprising an adapter device according to the invention for a disassemblable shaft instrument according to the invention,

FIG. 3 shows an exploded perspective view of the adapter device according to the invention,

FIG. 4 shows a longitudinal sectional view of the adapter device according to the invention in a coupling arrangement with a shaft and a transmission rod,

FIG. 5 shows a longitudinal sectional view of the adapter device according to the invention from FIG. 4 without a transmission rod and with a flushing device.

The invention relates to an adapter device for a disassemblable medical shaft instrument and accordingly also to a disassemblable medical shaft instrument provided therewith. Furthermore, the invention comprises methods for cleaning the disassemblable medical shaft instrument and for assembling the adapter device in a disassemblable medical shaft instrument.

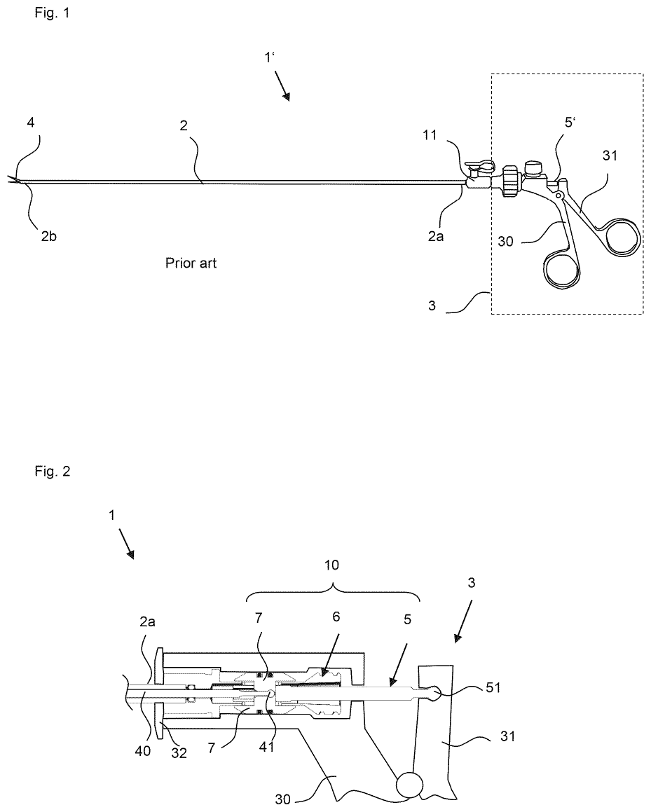

FIG. 1 shows a disassemblable medical shaft instrument 1′ from the prior art, which is composed of a handling apparatus 3, a shaft 2, and a tool insert comprising a tool 4 and a transmission rod (not visible in FIG. 1). The tool 4 is arranged at the distal end 2b of the shaft 2 so that the transmission rod extends through the shaft 2 and into the handling apparatus 3. At its proximal end 2a, the shaft 2 is releasably coupled to the housing part 30 of the handling apparatus 3, which apparatus comprises a conventional adapter device having a transmission adapter 5′, which is releasably coupled at one end to the proximal end of the transmission rod and at the other end to the actuating part 31 of the handling apparatus 3, which actuating part is movable relative to the housing part 30. By moving the actuating part 31 relative to the housing part 30, the actuating rod connected via the transmission adapter 5′ is moved in a translational manner within the shaft 2 to actuate the tool 4 at the distal end 2b of the shaft 2.

In the shaft instrument 1′ from the prior art, a flushing port 11 is arranged at the proximal end 2a of the shaft 2 at the interface with the handling apparatus 3. Due to the open design of the known transmission adapter 5′ described at the outset, a different flushing concept is not possible or does not lead to a sufficient flushing result.

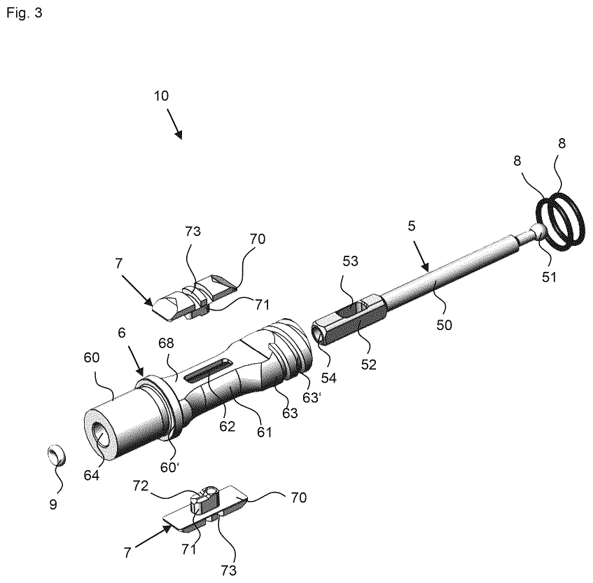

A disassemblable medical shaft instrument 1 according to the invention, shown in a sectional view in FIG. 2, differs from the conventional instrument 1′ in that it has an adapter device 10 according to the invention instead of the conventional transmission adapter 5′ and can therefore dispense with a flushing port 11 at the interface between the shaft 2 and the handling apparatus 3. As instrument components, a shaft instrument 1 according to the invention can comprise a shaft 2, a handling apparatus 3 and a tool 4 comprising a transmission rod 40 similarly to the conventional shaft instrument 1′ from existing series production. In this case, a shaft instrument according to the invention is not limited to the pliers tool 4 shown by way of example in FIG. 1, but can have any tool that can be actuated via a transmission rod.

As shown in FIG. 2, an adapter device 10 according to the invention connects the transmission rod 40 to the actuating part 31 by means of the transmission adapter 5 and the shaft 2 to the housing part 30 by means of the adapter sleeve 6, wherein the connection points can be releasable or non-releasable. The adapter device 1 thus enables a shaft 2 and a transmission rod 40 to be coupled to a handling apparatus 3 if the coupling elements of the transmission rod 40 and the shaft 2 do not correspond to the coupling elements of the actuating part 31 and the housing part 30, as may be the case, for example, with shafts under 5 mm and the matching tool inserts or transmission rods.

FIGS. 3 to 5 show that the adapter sleeve 6 for coupling to the housing part 30 has a distal end portion 60 which is cylindrical and delimited by a collar 60′. The collar 60′ serves, as indicated in FIG. 2, as a stop for arrangement in the housing part 30, which is connected at the distal end to a cover element 32 enclosing the shaft 2.

Additional coupling of the adapter sleeve 6 to the shaft 2 involves a distal portion 64 of the axial bore 64, 65, 66, 67 being designed to receive the proximal shaft end 2a. The proximal end 2a of the shaft 2 is received as far as a stop formed by a shoulder to a connecting portion 65 of the axial bore 64, 65, 66, 67 of reduced diameter, which connects to the distal portion 64. The connecting portion 65, which is designed for the passage of the transmission rod 40 and opens into a central portion 66 of the axial bore 64, 65, 66, 67, has a sealing groove 65′ adjacently to the distal portion 64, in which a sealing ring 9 is arranged to seal the transmission rod 40.

The transmission adapter 5 is used to connect the actuating rod 40, whose proximal coupling element 41 has a smaller diameter than the coupling receptacle (not designated in FIG. 2) of the actuating part 31. The transmission adapter 5 has a coupling element 51 at the proximal end of a rod portion 50, which coupling element can be received in the coupling receptacle of the actuating part 31.

In the example shown, the proximal coupling element 41 of the transmission rod 40 and the proximal coupling element 51 of the transmission adapter 5 are each designed like a ball head. However, modifications to the adapter device for modified shaft instruments are also conceivable, whose coupling elements have a different design.

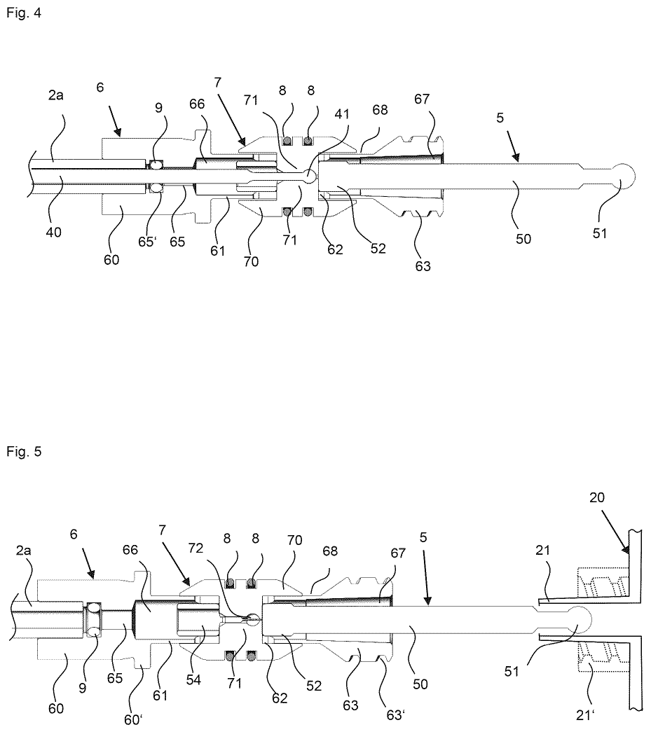

The translationally movable transmission adapter 5 extends by means of its distal coupling portion 52 into the axial bore 64, 65, 66, 67 of the adapter sleeve 6. In the enlarged view in FIGS. 4 and 5, it can be seen that the proximal portion 67 of the axial bore 64, 65, 66, 67 of the adapter sleeve 6 widens in a frustoconical shape toward the proximal end (on the right in the drawings).

The shape and dimensions of this conical portion 67 match those of a conical nozzle 21 of a flushing device 20, as is indicated in FIG. 5. By means of the conical portion 67, which matches the conical nozzle 21, a sealed, clamping plug connection in the form of the “Luer Slip” system is achieved when the nozzle 21 is received in the conical portion 67, which allows the axial supply of a rinsing medium from the proximal side of the adapter device 10. Said medium passes the transmission adapter 5 and enters the axial bore 64, 65, 66, 67 of the adapter sleeve 6, where it enters the shaft 2, the proximal end 2a of which is received in a distal portion 64 of the axial bore 64, 65, 66, 67 of the adapter sleeve 6.

In the present example, an external thread 63′ is formed on the proximal end portion 63 of the adapter sleeve 6, which is designed for threaded engagement with an internally threaded tube portion 21′ of the flushing device 20, which surrounds the nozzle 21. The threaded engagement secures the sealing plug connection according to the “Luer Lock” system and therefore also allows for higher pressures when supplying the flushing medium.

For coupling the transmission rod 40 to the transmission adapter 5, the adapter device 10 has two engagement slides 7, as can also be seen in FIG. 3. For this purpose, an axial receiving bore 54 and two engagement openings 53 that are opposite in the radial direction are formed in the distal coupling portion 52 of the transmission adapter 5. For this purpose, the adapter sleeve 6 has two opposite longitudinal openings 62 in a narrower central portion 61, which are associated with a central portion 66 of the axial bore 64, 65, 66, 67.

The distal coupling portion 52 of the transmission adapter 5 is placed in the central portion 66 of the adapter sleeve 6 such that the engagement openings 53 in the distal coupling portion 52 are aligned with the longitudinal openings 62 in the adapter sleeve 6. For axial guidance with respect to the adapter sleeve 6, the distal coupling portion 52 of the transmission adapter 5 can have a square shape.

The transmission rod 40, which is inserted from the distal side, is inserted by means of its proximal coupling element 41 into the axial receiving bore 54 until the coupling element 41 is located in the region between the engagement openings 53. Both engagement slides 7 are inserted by means of each of their plug portion 71 from both sides through each longitudinal opening 62 and the aligned engagement opening 53 into the axial receiving bore 54, and receive the proximal coupling element 41 of the transmission rod 40, as can be seen in FIG. 4, in a form fit in each recess 72 in the plug portion 71. The longitudinal openings 62 are longer than the plug portion 71 so that the engagement slide 7 connected to the transmission adapter 5 can follow its translational movement relative to the adapter sleeve 6. For safe guidance, the width of the plug portion 71-except for the play that is of course required for relative movement-corresponds to the width of the longitudinal opening 62.

In the example shown, the proximal coupling element 41 is designed like a ball head at the proximal end of the transmission rod 40 so that the recess 72 is accordingly shaped as a ball head receptacle. The size of the engagement opening 53 corresponds to the cross section of the plug portion 71, which is thus positively locked in the engagement opening 53. To facilitate assembly, the axial receiving bore 54 is of such a size that the proximal coupling element 41 is placed near the bottom of the bore for easy reception in the recess 72 of the plug portion 71.

In order to seal the adapter sleeve 6 in the region of the longitudinal openings 62, each engagement slide 7 has a slider portion 70 with a flat inner surface, from which the plug portion 71 extends orthogonally. The adapter sleeve 6 is formed in the central portion 61 around the longitudinal opening 62 with a flat sliding surface 68 on which the slider portion 70 rests by means of its flat inner side such that it can move in a translational manner. The length and width of the slider portion 70 are of such a size that the longitudinal opening 62 is always covered by the slider portion 70, i.e., even when the plug portion 71 strikes the proximal or distal end of the longitudinal opening 62.

To secure the engagement of the engagement slides 7, these have two circumferentially extending receiving grooves 73 on the outer side of the slider portion 70 in the example shown (cf. FIG. 3), in each of which grooves an elastic O-ring 8 is received, which, as can be seen in FIGS. 4 and 5, surrounds the central portion 61 of the adapter sleeve 6 to hold the two engagement slides 7 together.

The O-rings 8 allow for movement of the engagement slides 7 in the radial direction, which allows the tool insert comprising the tool 4 and the transmission rod 40 to rotate during assembly, e.g., for coupling the tool 4 to the distal shaft end 2b by means of a bayonet connection. When the disassemblable shaft instrument 1 is assembled, a receiving contour in the housing part 30, as indicated in FIG. 2, prevents the engagement slides 7 from being able to lift. Therefore, it is not possible to disassemble the tool insert when the disassemblable shaft instrument 1 is assembled or during use.

Even in the disassembled state of the disassemblable shaft instrument 1, i.e., without the handling apparatus 3 and without the tool insert consisting of the tool 4 and transmission rod 40, the O-rings 8 press the engagement slides 7 onto the sliding surfaces 68 and thus close the longitudinal openings 62 so that the flushing medium can only escape at the proximal end to a limited extent. Consequently, the flushing medium continues to be conveyed through the axial bore 64, 65, 66, 67 and through the shaft 2 to its distal end 2b.

The adapter device according to the invention, which dispenses with a joint-and-spring connection, allows for the construction of medical shaft instruments with an axial supply of a rinsing medium for minimally invasive instruments, which can in particular also have shaft diameters of less than 5 mm. This allows for optimized flushing in the proximal region of the adapter device used, without the need for an additional flushing adapter or other components, since the adapter device itself is sealed as a result of its construction. By integrating the flushing port into the proximal end portion of the adapter sleeve, reprocessing of the disassemblable shaft instruments can be optimized. The simple design of the adapter device with few components and simple contours without undercuts and few covered surfaces also makes it possible to dispense with permanent joining techniques such as welding or gluing, or at least allows them to be minimized.

The present invention provides an adapter device and a disassemblable medical shaft instrument equipped therewith, a method for cleaning the disassemblable medical shaft instrument and a method for assembling the adapter device to provide the disassemblable medical shaft instrument. The adapter device 10 is designed for arrangement in a disassemblable medical shaft instrument 1 comprising a shaft 2, a transmission rod 40 that is movable in the shaft 2 and a handling apparatus 3 comprising a housing part 30 and an actuating part 31 which is movable relative to the housing part 30. The adapter device 10 has a transmission adapter 5, which is designed for releasably coupling to the actuating part 31 and to the transmission rod 40, and an adapter sleeve 6, which is designed for releasably coupling to the shaft 2 and to the housing part 30. The adapter sleeve 6 has an axial bore 64, 65, 66, 67 in which a distal coupling portion 52 of the transmission adapter 5 is arranged, and wherein the transmission adapter 5 is movable in a translational manner relative to the adapter sleeve 6. According to the invention, a proximal portion 67 of the axial bore 64, 65, 66, 67 of the adapter sleeve 6 is frustoconical and widens toward the proximal end of the axial bore 64, 65, 66, 67, wherein the frustoconical proximal portion 67 is designed to releasably receive a conical nozzle 21 of a flushing device 20. The drawings, the description, and the claims contain numerous features in combination. It goes without saying that the above-mentioned features can be used not only in the combination specified in each case, but also in other combinations or on their own, without departing from the scope of the present invention.

### LIST OF REFERENCE SIGNS

1, 1′ Disassemblable shaft instrument
2 Shaft
2a, 2b Proximal, distal (shaft) end
3 Handling apparatus
4 Tool
5,5′ Transfer adapter
6 Adapter sleeve
7 Engagement slide
8 Securing O-ring
9 Sealing O-ring
10 Adapter device
11 Flushing port according to the prior art
20,21 Flushing device, conical nozzle
30, 31, 32 Housing part, actuating part, cover element
40,41 Transmission rod, proximal coupling element/ball head
50,51 Rod portion, proximal coupling element/ball head
52 Distal coupling portion
53 Engagement opening
54 Receiving bore
60,60′ Distal end portion, collar
61 Middle portion
62 Longitudinal opening
63, 63′ Proximal end portion, external thread
64 Distal portion of the axial bore
65,65′ Connecting portion of the axial bore, sealing groove
66 Middle portion of the axial bore
67 Proximal conical portion of the axial bore/
68 Sliding surface
70 Slider portion
71 Plug portion
72 Recess
73 Receiving groove

1. An adapter device, configured for arrangement in a disassemblable medical shaft instrument comprising a shaft, a transmission rod that is configured to move within the shaft and a handling apparatus comprising a housing part and an actuating part that can move relative to the housing part, the adapter device comprising:
a transmission adapter configured to releasably couple to the actuating part and to the transmission rod, and
an adapter sleeve configured to releasably or non-releasably couple to the shaft and to the housing part,
the adapter sleeve having an axial bore in which a distal coupling portion of the transmission adapter is arranged, and the transmission adapter being movable in a translational manner relative to the adapter sleeve,
wherein
a proximal portion of the axial bore of the adapter sleeve is frustoconical and widens toward the proximal end of the axial bore the frustoconical proximal portion being designed to releasably receive a conical nozzle of a flushing device.

2. The adapter device according to claim 1,
wherein
a proximal end portion of the adapter sleeve has an external thread which is configured to releasably connect to an internally threaded tube portion of the flushing device that surrounds the nozzle.

3. The adapter device according to claim 1,
wherein
the proximal frustoconical proximal portion is shaped in accordance with a standardized connecting system.

4. The adapter device according to claim 1,
wherein
the distal coupling portion of the transmission adapter has an axial receiving bore and at least one engagement opening in the radial direction that is associated with said bore, and
the adapter device has at least one engagement slide with a plug portion which is arranged in the engagement opening and extends into the receiving bore of the transmission adapter and has a recess which is designed to receive a proximal coupling element of the transmission rod,
the adapter sleeve having at least one longitudinal opening in a central portion that is associated with the axial bore and is longer than the plug portion which extends through the longitudinal opening for arrangement in the engagement opening of the distal coupling portion, the engagement slide together the transmission adapter being movable in a translational manner relative to the adapter sleeve.

5. The adapter device according to claim 4,
wherein
the engagement slide has a slider portion from which the plug portion extends, the adapter sleeve having a sliding surface in the central portion around the longitudinal opening on which the slider portion of the engagement slide rests such that it moves in a translational manner, the length and width of the slider portion being of such a size that the longitudinal opening is always covered by the slider portion.

6. The adapter device according to claim 4,
wherein
the adapter device has two engagement slides, two engagement openings in the distal coupling portion of the transmission adapter and two longitudinal openings in the central portion of the adapter sleeve, which are each opposite one another in the radial direction.

7. The adapter device according to claim 4,
wherein
the engagement slide has at least one circumferentially extending receiving groove on an outer side facing away from the plug portion, in which groove an elastic O-ring is received, which surrounds the central portion of the adapter sleeve.

8. The adapter device according to claim 1,
wherein
the distal coupling portion has an edge shape with at least one axially parallel guide surface for the axial guidance of the transmission adapter in the adapter sleeve.

9. The adapter device according to claim 1,
wherein
a distal portion of the axial bore is configured to releasably or non-releasably receive a proximal end of the shaft and is associated with a connecting portion of the axial bore of reduced diameter, which is configured for the passage of the transmission rod,
and/or
a distal end portion of the adapter sleeve is cylindrical and is limited by a collar which is a stop for arrangement in the housing part.

10. A disassemblable medical shaft instrument comprising:
a handling apparatus having a housing part and an actuating part configured to move relative thereto, a shaft, a transmission rod that can move in the shaft and an adapter device comprising a transmission adapter for releasably coupling to the actuating part and to the transmission rod, and an adapter sleeve for releasably or non-releasably coupling to the housing part and to the shaft,
a distal coupling portion of the transmission adapter being arranged in an axial bore of the adapter sleeve and the transmission adapter being movable in a translational manner relative to the adapter sleeve
wherein
the adapter device is an adapter device according to claim 1.

11. A method for cleaning a disassemblable medical shaft instrument comprising:
decoupling a transmission adapter from an actuating part and decoupling an adapter sleeve from a housing part, and
separating the transmission adapter that is coupled to a transmission rod and the adapter sleeve that is coupled to the shaft from the handling apparatus,
decoupling the transmission adapter from the transmission rod and removing the transmission rod from the shaft,
arranging a conical nozzle of a flushing device in a frustoconical proximal portion of the axial bore of the adapter sleeve, and
supplying a flushing medium by means of the flushing device through the nozzle into the axial bore of the adapter sleeve and the shaft coupled thereto, thereby cleaning the medical shaft instrument.

12. A method for assembling an adapter device for providing a disassemblable medical shaft instrument
comprising:
providing a transmission adapter and an adapter sleeve and providing a handling apparatus comprising a housing part and an actuating part that can move relative thereto and the shaft with a transmission rod that can move in the shaft,
arranging a distal coupling portion of the transmission adapter in an axial bore of the adapter sleeve,
releasably coupling the transmission adapter to the transmission rod and releasably or non-releasably coupling the adapter sleeve to the shaft,
arranging the transmission adapter that is coupled to the transmission rod and the adapter sleeve that is coupled to the shaft in the handling apparatus,
releasably coupling the adapter sleeve to the housing part and releasably coupling the transmission adapter to the actuating part.

13. The method according to claim 12, wherein the releasable coupling between the transmission adapter and the transmission rod further comprises:
aligning the at least one engagement opening associated with the axial receiving bore in the distal coupling portion with the at least one longitudinal opening in the central portion of the adapter sleeve when arranging the distal coupling portion of the transmission adapter in the axial bore of the adapter sleeve arranging the proximal coupling element of the transmission rod in the receiving bore in the distal coupling portion,
providing the at least one engagement slide,
inserting the plug portion of the engagement slide through the longitudinal opening and the engagement opening and into the receiving bore, and
receiving the proximal coupling element of the transmission rod in the recess in the plug portion.

14. The method according to claim 13, wherein the releasable coupling between the transmission adapter and the transmission rod further comprises:
arranging an elastic O-ring in the receiving groove on the outside of the el-öä-ö.ngagement slide
